(19) United States
(12) Patent Application Publication
(10) Pub. No.: US 2006/0041189 A1
(43) Pub. Date: Feb. 23, 2006

(54) Grummet

(76) Inventor: Thierry G. Vancaillie, Castlecrag (AU)

Correspondence Address: JAMES EPPA HITE, III, 2318 LOUIS ROAD, PALO ALTO, CA, 94303-3635, US

(21) Appl. No.: 11/205,461

(22) Filed: Aug. 16, 2005

Related U.S. Application Data
(60) Provisional application No. 60/602,741, filed on Aug. 17, 2004.

(51) Int. Cl. A61B 1/00

(52) U.S. Cl. 600/154

(57) ABSTRACT

A grummet, for covering the end of an instrument channel of an endoscope, has a cone-shaped surface providing an entrance and forming an anti-reflux valve.

This application claims the benefit of U.S. Provisional Application No. 60/602,741, filed Aug. 17, 2004.

### BACKGROUND OF THE INVENTION

1. Field of the Invention

The present invention relates generally to endoscopes and more particularly to a grummet used to cap the end of an instrument channel of an endoscope.

2. Discussion of Prior Art

Endoscopy, such as hysteroscopy, gastroscopy, cystoscopy, arthroscopy and so on, has rapidly expanded during the past century. As early as 1920 the basic endoscope with a visual channel was improved with the addition of an instrument channel designed to pass rigid instruments. The main objective was to obtain tissue samples for histopathology or microbiology. If the instrument channel is open then fluid can escape from the fluid filled system of an organ to be examined. To close off the instrument channel, a grummet and stopcock were used. A conventional grummet resembles a simple cap. It has a flat end with a rigid central opening, large enough to allow a rigid ancillary instrument, such as a guide wire or a pair of scissors, to pass through without much difficulty, but fitting snugly around the instrument to close the fluid-filled system. The stopcock is also for preventing leakage of fluid from the system, which is necessary to obtain distention of the organ to be examined endoscopically.

More recently developed catheter based instruments are flexible and not as sturdy as their rigid metal predecessors. Examples are the instruments used for female sterilization. They are fitted with a flexible tip which is easily damaged and, due to lack of rigidity, does not pass through the opening of a conventional flat-ended grummet. Flexible catheter instruments introduced through the grummet are likely to buckle. To overcome this difficulty, a so-called “introducer” consisting of a metal rod surrounded by a plastic sheath is passed through the flat grummet to overcome the resistance of the round opening. The rod is then withdrawn, leaving the plastic sheath within the opening of the grummet. Next, the soft flexible catheter is threaded through the plastic sheath into the instrument channel. This maneuver is cumbersome, time-consuming and sometimes forgotten, resulting in the loss of a valuable catheter.

### SUMMARY OF THE INVENTION

The present invention provides a grummet for the instrument channel of an endoscope. A grummet according to a preferred embodiment is a partly hollow cylinder. The proximal end of the grummet fits on the end of the working channel of an endoscope. The distal end is shaped as an inverted cone, pointing inside the hollow cylinder towards the working channel.

The cone-shaped distal surface facilitates the passage of flexible instruments. An instrument being forced through the hollow of the inverted cone will part the two surfaces of the hole at its tip, which forms a flap valve. The inner walls of the cone provide sufficient column strength to a flexible catheter to give it the necessary rigidity to pass the flap valve mechanism.

Further, the tip of the cone forming an anti-reflux flap valve obviates the need for a stopcock in the instrument channel of the endoscope. Fluid filling the endoscope system and surrounding the conical surface which faces the hollow of the grummet will pressure the two opposing surfaces of the bicuspid flap valve into the closed position.

These and other advantages of the invention will become apparent to those skilled in the art upon reading the following detailed description of the preferred embodiment as illustrated in the several figures of the drawing.

### BRIEF DESCRIPTION OF THE DRAWING

FIG. 1 is a perspective view of a grummet according to the invention;

FIG. 2 shows a section AA at a plane seen in direction A-A of FIG. 1 through the middle of the grummet;

FIG. 3 details area C of FIG. 2, showing the flap valve mechanism of the opening of the grummet; and

FIG. 4 is a cross-section of a grummet, in an embodiment having a longer conical portion than that of FIG. 2, inserted in the end of an endoscope and receiving the tip of an instrument.

### DETAILED DESCRIPTION OF THE INVENTION

The present invention avoids problems of needing a stopcock and an introducer through providing a cone-shaped surface for a grummet. The vertex of the cone points towards the inside of the instrument channel, and at its tip has an opening in the form of a flap valve. The flap valve shape prevents fluid on the larger side of the cone from escaping the instrument channel, thereby obviating the need for a stopcock. The funnel-like entrance to the central hole on the smaller side of the cone guides flexible instruments, thereby obviating the need for an introducer to open the grummet hole.

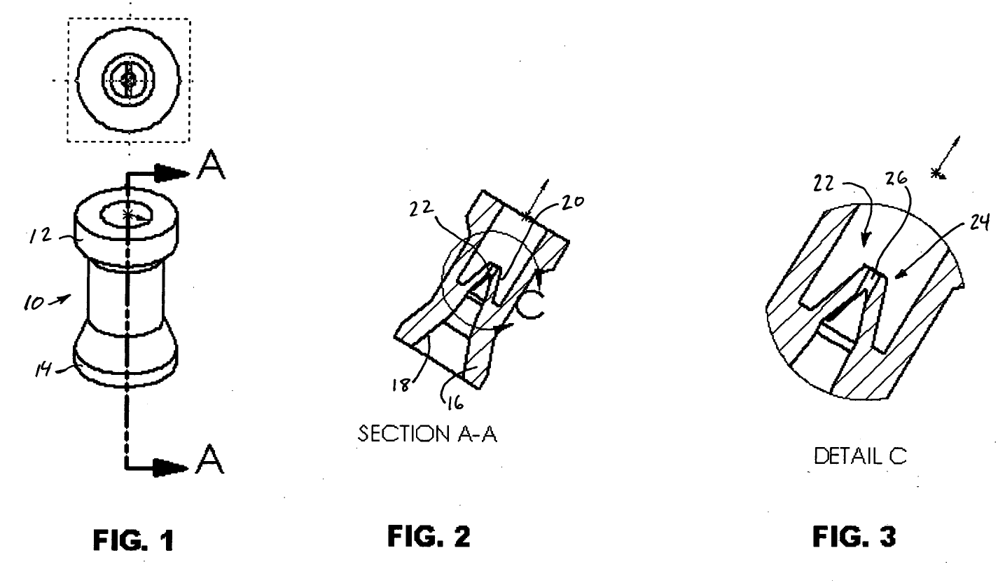

FIG. 1 is a perspective view of a grummet according to the invention. It may be formed in one piece. It has a basically cylindrical shaped hollow body 10. The proximal end 12 and distal end 14 may flare our as shown.

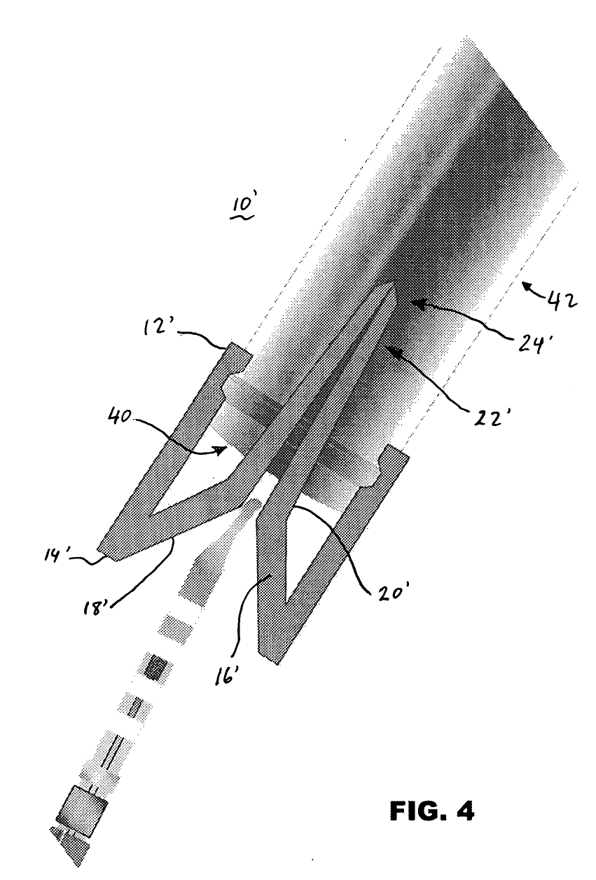

FIG. 2 shows a section AA rotated at a plane seen in direction A-A of FIG. 1 through the middle of the grummet. Inside body 10 the proximal end 12 is hollow and has an inside diameter suitable to cap the end of the instrument channel of an endoscope sheath. The distal end 14 forms a conical portion 16 having a base in common with the distal end of the cylindrical body and a vertex directed towards the proximal end. Inverted conical portion 16 has an inner (upstream) surface 18 and a outer (downstream) surface 20. Inner surface 18 is preferably approximately 1 cm long (but could be longer as in FIG. 4, although with little added benefit). This provides support for the flexible catheter when passed along surface 18 through the grummet. Within the middle of the grummet, the tip 22 of the conical surface represents the transition between the outside and the instrument channel, corresponding to the central opening of the traditional grummet.

FIG. 3 details area C of FIG. 2, showing the tip 22 having a flap valve mechanism 24 forming the opening 26 of the grummet.

Referring to FIG. 4 a grummet in a second embodiment 10′ is shown attached to the opening 40 of the instrument channel 42 of an endoscope (not shown). Bicuspid flap valve 24′ points toward the endoscope. The outer (downstream) surface 20′ of the flap valve mechanism is situated within the hollow of the proximal part 12′ of the grummet which is in continuity with the lumen of the instrument channel 42. Fluid contained within the lumen of the instrument channel of the endoscope fills the space around the flap valve and thereby creates a pressure which will keep the valve 24′ closed. Thus the flap valve remains closed and prevents fluid from leaking out of the system. On the other hand, however, a catheter passed through the valve 24′ will easily part the bicuspid valve and be allowed to pass into the instrument channel 42.

While the present invention is described in terms of a preferred embodiment, it will be appreciated by those skilled in the art that this embodiment may be modified without departing from the essence of the invention. It is therefore intended that the following claims be interpreted as covering any modifications falling within the true spirit and scope of the invention.

I claim:

1. A grummet, comprising:
a cylindrical portion having a wall forming a hollow cylinder; and
a hollow conical portion having a wall with an inside surface and an outside surface, a base coincident with the wall of the cylindrical portion, and at its vertex a valve with an opening that can be held closed by fluid pressure on the outside surface, or spread open by the insertion of a flexible catheter on the inside surface.
